(19) United States
(12) Patent Application Publication
(10) Pub. No.: US 2018/0177540 A1
(43) Pub. Date: Jun. 28, 2018

(54) METHOD FOR CONTROLLING THE VISCOSITY OF ORTHOPEDIC BONE CEMENT

(71) Applicants: UNIVERSITÉ DE STRASBOURG, Strasbourg (FR); CENTRE NATIONAL DE LA RECHERCHE SCIENTIFIQUE, Paris (FR); INSTITUT NATIONAL DES SCIENCES APPLIQUÉES, Strasbourg (FR)

(72) Inventors: Nicole LEPOUTRE, Strasbourg (FR); Laurence MEYLHEUC, Bergbieten (FR); Gabriela Iuliana BARA, Illkirch (FR); Bernard BAYLE, Strasbourg (FR)

(73) Assignees: UNIVERSITÉ DE STRASBOURG, Strsbourg (FR); CENTRE NATIONAL DE LA RECHERCHE SCIENTIFIQUE, Paris (FR); INSTITUT NATIONAL DES SCIENCES APPLIQUÉES, Strasbiurg (FR)

(21) Appl. No.: 15/739,884

(22) PCT Filed: Jun. 24, 2016
(86) PCT No.: PCT/EP2016/064738

§ 371 Date: Dec. 26, 2017

(30) Foreign Application Priority Data
Jun. 24, 2015 (EP) 15305975.3

(51) Int. Cl. A61B 17/88; G05D 24/00

(52) U.S. Cl. CPC A61B 17/8836; A61B 2017/00022; G05D 24/00

(57) ABSTRACT

Some embodiments are directed to a method for controlling the viscosity of orthopedic bone cement during its curing in percutaneous vertebroplasty by allowing a controlled heating and/or cooling of the cement during the injection that leads to a dynamic and full control of the viscosity of the cement during the injection.

### CROSS REFERENCE TO RELATED APPLICATION(S)

This application is national phase filing under 35 C.F.R. § 371 of and claims priority to PCT Patent Application No. PCT/EP2016/064738, filed on Jun. 24, 2016, which claims the priority benefit under 35 U.S.C. 119 of European. Patent Application No. 15305975.3, filed on Jun. 24, 2015, the contents of each of which are hereby incorporated in their entireties by reference.

### BACKGROUND

Some embodiments relate to a method for controlling the viscosity of orthopedic bone cement during its curing in percutaneous vertebroplasty. Some embodiments also relate to an injection device that allows the control.

Percutaneous vertebroplasty is a non-surgical minimal invasive intervention that involves injecting bone cement into the vertebral body of a patient, under medical imaging control, to reinforce or restructure a weakened or broken vertebra. Such an intervention allows the stabilization of the vertebrae and the reduction of severe pain for people suffering from vertebral compression fractures, most often caused by osteoporosis but also, less frequently, by metastases or traumatic fractures^{[1]}.

During a vertebroplasty intervention, patients are generally placed in prone position under conscious sedation (mild sedation and analgesia). Depending on the size of each pathological vertebrae and its level of inside damage, the practitioner inserts one or two bone trocars via a transpedicular approach using Computed Tomography (CT) or fluoroscopic guidance. Once the trocars inserted, the orthopedic bone cement is typically prepared (mixing phase) by mixing a powder based on polymethylmethacrylate (PMMA) and a liquid form of methylmethacrylate (MMA). To promote the exothermic free radical polymerization process, the powder also incorporates an initiator while the liquid includes an activator. In order to obtain a radio-opaque cement, a radiopacifier figures also in the powder. The mixing phase ends when the cement is homogeneous. Then, a waiting phase occurs until its viscosity achieves a minimum threshold, which is left to the decision of the physician. Indeed, the operator draws on his experience to know when the cement is ready to be injected, which is a highly subjective diagnosis. This phase can last one or two minutes. Afterwards, the radiologist fills the conventional syringe or the delivery device and plugs it to the inserted needle. The cement injection occurs at that time under continuous radioscopic control in order to identify any potential leaks. This working phase (or hardening phase) does not last more than 10 to 15 minutes since afterwards the cement becomes too viscous to be injected.

### SUMMARY

Despite fast and significant benefits from the patient's perspective, this procedure introduces two high-risk sources:
firstly, defined as a postoperative complication, the risk of cement leakage outside the damaged vertebra is critical since the consequences might be dramatic. Indeed, the patient may end up with a pulmonary embolism due to cement leakage. This risk is all the more significant given that the bone cement has a low viscosity at the beginning of the injection;
secondly, the practitioner's permanent exposure to harmful X-rays causes adverse effects on his health.

The aim of this invention is therefore to remedy to the first above-mentioned drawbacks, notably to avoid or at least to reduce this risk of cement leakage. In that context, the Applicant has now developed a method for controlling the viscosity of orthopedic bone cement during its curing by acting on the bone cement temperature in percutaneous vertebroplasty. The Applicant has also developed an injection device (not claimed) for implementing the control during a vertebroplasty intervention, to remedy to the second above-mentioned drawback.

One of oridnary skill in the art knows methods involving the cooling or the heating of the cement during the intervention. However, in these known methods, the cooling of the cement is limited to a pre- or per-operative conservation of its fluidity before the actual injection^{[2], [3]}.

Furthermore, it is also known by one of ordinary skill in the art to use heating of the cement to increase its viscosity^{[4]} but the heating is not dynamically controlled. At last, even if the injection device of U.S. Pat. No. 8,523,871^{[4]} implements both heating and cooling functions, only the actual heating can be controlled because of the positioning of the sensor.

Some embodiments are therefore directed to a method for controlling the viscosity of orthopedic bone cement during its curing in percutaneous vertebroplasty that prevents both aforementioned drawbacks, notably by allowing a controlled heating and/or cooling of the cement during the injection that leads to a dynamic and full control of the viscosity of the cement during the injection.

In the method taught by U.S. Pat. No. 8,523,871^{[4]} the principle can include or can consist of using a slow curing bone cement and to manage its viscosity at the entrance of the vertebra via a radiofrequency energy. As a result, leakage risks consistently decrease. However, the drawbacks of such a method are that the physician has to know how to use RF pulse and this method is cement-dependent. At last, temperatures inside the vertebra may reach values up to 200° C., which adds possible complications towards tissue neighboring the damaged vertebra.

Unlike the method taught by U.S. Pat. No. 8,523,871^{[4]}, the method of some embodiments allows a precise, dynamic and full control of the temperature of the cement in a given section of the pipe in order to follow a viscosity set point η*, evolving over time in a given interval [ηₘᵢₙ, ηₘₐₓ]. The control of the bone cement temperature can include or can consist of:
accelerating the curing reaction by heating the viscosity of the cement is lower than η*;
slowing down the curing reaction the cooling if the viscosity of the cement is higher than η* (in U.S. Pat. No. 8,523,871[4], even if both heating and cooling functions are implemented, only the heating can be controlled).

Some embodiments are directed to a method for the dynamic control of the viscosity of orthopedic bone cement during its curing by acting on the bone cement temperature in percutaneous vertebroplasty, within an injection device including a syringe, a percutaneous needle connected to the syringe via a pipe, including an active heat exchanger, the method including:

A. defining the time tₒ, time at which the radiologist starts the mixing process of the bone cement;

B. filling the syringe with the prepared bone cement;

C. defining for the bone cement a target viscosity η* to be reached or maintained, the target viscosity η* being included in the range [ηₘᵢₙ−ηₘₐₓ], ηₘᵢₙ being the minimal threshold viscosity of the cement which has to be reached for beginning the injection and ηₘₐₓ being the maximum threshold viscosity of the cement above which the injection is not possible anymore;

D. beginning the injection of the bone cement into the vertebra;

E. at instant t during the injection:
e1) measuring the effective temperature T of the bone cement at the outlet of the active heat exchanger and, possibly, measuring the effective temperature Tᵢ of the bone cement at its inlet;
e2) computing the pressure drop ΔP=Pₒ−Pᵢ along the pipe between the outlet of the syringe and a given intermediate point, Pₒ being the pressure measured at the outlet of the syringe and Pᵢ being the pressure measured at the given intermediate point on the pipe, the length between those two points being denoted as Lₛₑₙₛₒᵣ;
e3) computing the flow rate Q of the bone cement in the pipe;
e4) computing the shear rate \dot{γ}ₚ at the wall of the pipe as a function of the flow rate Q, the cross-section dimensions of the pipe and the intrinsic physical parameters of the cement;
e5) calculating the instant viscosity η(t,T,\dot{γ}_{P}) if Q is nonzero, as a function of time t, temperature T, pressure drop ΔP and shear rate \dot{γ}ₚ, itself function of the flow rate Q;

η₀(t,T) if Q has a zero value, as a function of time t and temperature T;
e6) computing a set point temperature T*(t) associated to the target viscosity η* and the instant viscosity η, η* being function of the flow rate Q and the time t;
e7) calculating the difference ε_{T} between the previously determined set point temperature T*(t) and the effective temperature at the outlet of the heat exchanger T;
e8) controlling the cooling or the heating of the bone cement throughout the control of the active heat exchanger as a function of ε_{T}:

F. at instant t+Δt, redefining possibly the target viscosity η* in the range [ηₘᵢₙ: ηₘₐₓ] and repeating step E until the end of the injection, unless the instant viscosity η(t,T,\dot{γ}_{P}) and/or η₀(t, T) has reached the maximum threshold viscosity ηₘₐₓ.

By measuring Pᵢ at the given intermediate point on the pipe, it is meant in the sense of some embodiments, either a physical measurement or it is supposed to be the atmospheric pressure.

Some embodiments are also directed to a method for the dynamic control of the viscosity of orthopedic bone cement during its curing by acting on the bone cement temperature in percutaneous vertebroplasty, within an injection device including a syringe, a percutaneous needle connected to the syringe via a pipe, including an active heat exchanger, the method including:
A. defining the time tₒ, time at which the radiologist starts the mixing process of the bone cement;
B. filling the syringe with the prepared bone cement;
C. defining for the bone cement a target viscosity η* to be reached or maintained, the target viscosity η* being included in the range [ηₘᵢₙ−ηₘₐₓ], ηₘᵢₙ being the minimal threshold viscosity of the cement which has to be reached for beginning the injection and ηₘₐₓ being the maximum threshold viscosity of the cement above which the injection is not possible anymore;
D. beginning the injection of the bone cement into the vertebra;
E. at instant t during the injection:
   e1) measuring the effective temperature T of the bone cement at the outlet of the active heat exchanger and, possibly, measuring the effective temperature Tᵢ of the bone cement at its inlet;
   e2) computing the pressure drop ΔP=Pₒ−Pᵢ along the pipe between the outlet of the syringe and a given intermediate point, Pₒ being the pressure measured at the outlet of the syringe and Pᵢ being the pressure measured at the given intermediate point on the pipe, the length between those two points being denoted as Lₛₑₙₛₒᵣ;
   e3) computing the flow rate Q of the bone cement in the pipe;
   e4) computing the shear rate \dot{γ}ₚ at the wall of the pipe as a function of the flow rate Q, the cross-section dimensions of the pipe and the intrinsic physical parameters of the cement;
   e5) calculating the instant viscosity
η(t,T,\dot{γ}_{P}) if Qis nonzero, as a function of time t, temperature T, pressure drop ΔP and shear rate \dot{γ}ₚ, itself function of the flow rate Q;
η₀(t,T) if Q has a zero value, as a function of time t and temperature T;
   e6) computing a set point temperature T*(t) associated to the target viscosity η* and the instant viscosity η,η* being function of the flow rate Q and the time t;
   e7) calculating the difference ε_{T} between the previously determined set point temperature T*(t) and the effective temperature at the outlet of the heat exchanger T;
   e8) controlling the cooling or the heating of the bone cement throughout the control of the active heat exchanger as a function of ε_{T};
F. at instant t+Δt, repeating step E until the end of the injection, unless the instant viscosity η(t,T,\dot{γ}_{P}) and/or η₀(t,T) has reached the maximum threshold viscosity ηₘₐₓ.

In that case, step F may further include the redefinition of the target viscosity η* before repeating step E until the end of the injection, unless the instant viscosity η(t,T,\dot{γ}_{P}) and/or η₀(t, T) has reached the maximum threshold viscosity ηₘₐₓ.

By measuring Pᵢ at the given intermediate point on the pipe, it is meant in the sense of some embodiments, either a physical measurement or it is supposed to be the atmospheric pressure.

In the methods of some embodiments, the first step can include or can consist of defining the time tₒ, at which the radiologist starts the mixing process of the bone cement (step A). As indicated above, the mixing phase typically can include or can consist of mixing a powder based on polymethylmethacrylate (PMMA), an initiator and a radiopacifier, and a liquid form of methylmethacrylate (MMA) and an activator.

The mixing phase ends when homogeneous cement is reached. Afterwards, the radiologist fills the syringe, places it on the delivery device (not claimed) and plugs it to the inserted needle (step B).

Once the delivery device is ready for use, the cement injection (step D) occurs under continuous radioscopic control in order to identify any potential leaks.

In the normal course, the working phase does not last more than 10 to 15 minutes since afterwards the cement becomes too viscous to be injected and has reached a maximum threshold viscosity ηₘₐₓ above which the injection is not possible anymore. Moreover, as explained above, the risk of leakage outside the damaged vertebra is considerable since bone cement has a very low viscosity at the beginning of the injection. This brief stage leaves little time to the radiologist to perform his intervention.

Therefore, an advantage of the method of some embodiments for controlling the viscosity of the bone cement during its curing is twofold:
on the one hand, to accelerate the polymerization process until the bone cement reaches a viscosity ηₘᵢₙ that is the minimal threshold viscosity of the cement which has to be reached for beginning the injection, and
on the other hand, to minimize the viscosity evolution rate to increase the working phase.

Once the viscosity passes this threshold ηₘᵢₙ, the leakage risk is already highly reduced.

This lower viscosity limit is obtained by a preliminary study with the help of experimented radiologists. For this study, a physician prepares the bone cement. A small sample is collected and placed on the rotational rheometer while the rest of the mixture is poured out in the conventional injector. The expert is asked to inject slowly at a constant velocity so that the same shear rate can be applied on the sample on the dynamic rheometer. Once he estimates the minimum ηₘᵢₙ is attained, the viscosity value is read on the rheometer. Statistics on several trials with different physicians offer a good estimate ηₘᵢₙ. This can be done for all or most of the cements on the market.

The method of some embodiments necessarily includes a step C to define, for the bone cement, a target viscosity η* to be reached or maintained, that is included in the range [ηₘᵢₙ−ηₘₐₓ], ηₘᵢₙ being the minimal threshold viscosity of the cement which has to be reached for beginning the injection and ηₘₐₓ being the maximum threshold viscosity of the cement above which the injection is not possible anymore.

At instant t during the injection, the following measurements are made:
the effective temperature T of the bone cement (step e1) at the outlet of the active heat exchanger, and possibly, the effective temperature Tᵢ of the bone cement at its inlet, and
   the pressure Pₒ measured at the outlet of the syringe and the pressure Pᵢ at a given intermediate point on the pipe, thus giving the pressure drop ΔP=Pₒ−Pᵢ along the pipe between the outlet of the syringe and the given intermediate point (step e2).

According to a first implementation of the method of some embodiments, step e2) may be realized between the outlet of the syringe and the outlet of the needle.

According to a second implementation of the method of some embodiments, step e2 may be realized between the outlet of the syringe and the outlet of the active heat exchanger.

In that case, the intravertebral pressure P_{vertebra} may be computed according to formula (1):

$${P}_{vertebra} = {P}_{o} ( 1 - \frac{{L}_{vertebra}}{{L}_{sensor}} ) + \frac{{L}_{vertebra}}{{L}_{sensor}} {P}_{i} \qquad ( 1 )$$

with:
L_{vertebra} being the length included between the outlet of the syringe and the outlet of the needle.

Advantageously, the step of computing the flow rate Q of the bone cement in the pipe may include a step of measuring a moving speed Vₚᵢₛₜ of the piston of the syringe, the piston being driven to vary the volume of the cement in the syringe, the volumetric flow Q being then. given by Q=Vₚᵢₛₜ.π.r², where r is the radius of the pipe.

At instant t during the injection, besides the above-mentioned measurements the following calculations are also made:
the flow rate Q of the bone cement in the pipe (step e3)
the shear rate \dot{γ}ₚ at the wall of the pipe as a function of the flow rate Q, the cross-section dimensions of the pipe and the intrinsic physical parameters of the cement (step e4),
the instant viscosity (step e5) η(t,T,\dot{γ}_{P}) if Q is nonzero and or η₀(t, T) if Q has a zero value,
the set point temperature T*(t) associated to the target viscosity η* (step e6), and
the difference ε_{T} between the previously determined set point temperature T*(t) and the effective temperature at the outlet of the heat exchanger T (e7).

The instant viscosity η(t,T,\dot{γ}_{P}), if the flow rate is nonzero, may be calculated according to modified Power Law as defined by formula (2) in the case of a pipe having a cylindrical geometry of radius r:

$$η ( t , T , {\dot{γ}}_{P} ) = {a}_{{T}_{0}} ( T ) K ( t ) {( {a}_{{T}_{0}} ( T ) {\dot{γ}}_{P} )}^{n ( t ) - 1} \\ with \\ {a}_{{T}_{0}} ( T ) = exp ( \frac{- {E}_{a}}{R} ( \frac{1}{T} - \frac{1}{{T}_{0}} ) ) \qquad ( 2 )$$

with:
Eₐ being the activation energy in J.mol⁻¹,
T being the effective temperature of the bone cement at the outlet of the active heat exchanger,
T₀ being a reference temperature at which the viscosity η₀ is known,
R being the gas constant,
n(t) being the flow index of the bone cement at the current time t, n is either a known constant or defined as a function of t.
\dot{γ}ₚ being the shear rate at the wall of the pipe being given by formula (3):

$${\dot{γ}}_{P} = \frac{Q}{π {r}^{3}} \frac{3 n ( t ) + 1}{n ( t )} \qquad ( 3 )$$

with r being the radius of the pipe.
K(t) being given by formula (4):

$$Q = {( \frac{Δ P}{{L}_{sensor}} )}^{1 / n ( t )} {( \frac{r}{2 K ( t )} )}^{1 / n ( t )} ( \frac{π n ( t ) {r}^{3}}{3 n ( t ) + 1} ) \qquad ( 4 )$$

Advantageous the instant viscosity η(tT,\dot{γ}_{P}), whether for a flow rate being nonzero or having a zero value, may be calculated according to the differential equation (5):

\dot{η}(t,T\dot{γ}_{P})=f(η(t,T,\dot{γ}_{P})) (5)

where the time derivative \dot{η} of the viscosity is defined as a function the viscosity η, as taught by the publication of N. Lepoutre, G. Bara, L. Meylheuc, et B. Bayle, “Phase Space Identification Method for Modeling the Viscosity of Bone Cement”, in Control Conference (ECC), 2016 European, Juin-Juillet 2016^{[5]}.

Thus, during the injection (flow rate value being nonzero), it is thus possible to calculate the instant viscosity η(t,T,\dot{γ}_{P}) either according to equation (2) in combination with equations (3) and (4), or according to equation (5). However, when the injection is stopped (flow rate value being zero), the instant viscosity η₀(t,T) may only be calculated according to equation (5).

At the beginning of the injection, in order to reach as soon as possible the lower limit ηₘᵢₙ, it is possible or preferable to heat the PMMA. According to the results of an off-line characterization of bone cements, the higher the temperature of the cement is, the faster its viscosity increases. However, a runaway reaction is not excluded if the cement is warmed too much, so caution is a watchword. At the same time, reducing the injection flow can shorten the waiting time, since at low shear rates viscosity is higher.

As regards to the generation of the set point temperature T*, the most sensitive part of some embodiments lies in the injection time that has to be extended. On the opposite, the colder the cement stays, the longer the curing reaction lasts. Hence, to increase the injection time, it is possible or preferable to cool the bone cement. Note that it is difficult or impossible to completely stop the increase of viscosity. Its evolution can just be slowed down. This leads to the set point T* temperature.

Possibly, minimizing PMMA viscosity evolution can be expressed as:

min(\dot{η}(t,T,\dot{γ}_{P}))

Thus, the set point temperature T*(t) may be calculated according to a chosen control strategy either via

$${argmin }_{T} \dot{η}$$

or using the inverse solution of equation (5).

According to the method of some embodiments, the control (step e8) of the cooling or the heating of the bone cement is realized throughout the control of the active heat exchanger as a function of ε_{T}.

Possibly or preferably, the controlling e8) of the active heat exchanger may achieve the cooling or heating of the bone cement as a function of ε_{T} throughout a temperature regulation scheme composed of two nested closed-loops, where:
a temperature controller C_{T} uses the difference between the previously determined set point temperature T*(t) and the effective temperature T to compute the current reference I* of the active heat exchanger, the current reference I* being limited by a current saturation block,
a current controller C_{I} uses the difference ε_{I} between the current reference I* and the effective input current I to compute the input voltage U of a power supply H driving the active heat exchanger.

At instant t+Δt, the target viscosity η* may be redefined in the range [ηₘᵢₙ: ηₘₐₓ] (but not necessarily, as described above) and step E is repeated until the end of the injection, unless the instant viscosity η(t,T,\dot{γ}_{P}) and/or η₀(t, T) has reached the maximum threshold viscosity ηₘₐₓ.

Some embodiments are directed to an injection device of curing cement for percutaneous vertebroplasty, the device including a system for generating volumetric flow of the cement, and a pipe connecting the injection device to a percutaneous needle, the injection device being characterized in that it further includes at least one active heat exchanger(s) located on the pipe for dynamic controlled heating and/or cooling of the cement during the injection.

By active heat exchanger, it is meant according to some embodiments, a heat exchanger that operates in cooling or heating with. external energy and controls the heat exchanges.

Contrary to the teaching of U.S. Pat. No. 8,523,871^{[4]} (where even if both heating and cooling functions are implemented, only the heating can be controlled), the active heat exchanger of the injection device of some embodiments allows a precise, dynamic and full control of the temperature of the cement in a given section of the pipe in order to follow a target viscosity η*, evolving , evolvdng over time in a given interval [ηₘᵢₙ, ηₘₐₓ]. This control is achieved by:
accelerating the curing reaction by heating if the viscosity of the cement is lower than η*;
slowing down. the curing reaction by cooling if the viscosity of the cement is higher than η*.

As the cement itself acts as a thermal insulator, it is possible to regulate it on. reduced pipe diameters and therefore it is less convenient to do it on the syringe as claimed in the US patent application US2013/0190680^{[2]}.

The system for generating volumetric flow of the device of some embodiments may advantageously include a syringe for containing the cement, and a piston, that can move inside the syringe for pushing the cement inside the pipe through the syringe outlet.

According to a realization of some embodiments, the active heat exchanger(s) located along the pipe may include a thermal block with at least one Peltier module mounted on the pipe. Possibly or preferably, the thermal block may include:
a central regulated block made out of a thermal conducting material with low thermal inertia,
at least one stack on the regulated block and including:
   a Peltier module,
   at least one heat sink made out of thermal conducting material with low thermal inertia,
   a fan,
a thermal insulation wrapping the central block, to improve the efficiency of the heat exchanger, and
at least one temperature sensor.

According to another embodiment, the injection device may also further include a deported active heat exchanger put on a closed fluid circuit with a fluid-to-cement heat exchanger. The deported active heat exchanger of some embodiments operates in cooling and/or in heating, in order to reduce the exchanger's footprint at the proximity of the patient.

Possibly or preferably, the deported active heat exchanger of some embodiments may also include at least one Peltier module.

Advantageously, in this deported embodiment, the heat transfer fluid flowing between the deported active heat exchanger and the fluid-to-cement exchanger may be a liquid, a gas or a mixture of liquid and gas, and possibly or preferably water.

Besides the active heat exchanger(s) located. on the pipe, the injection device of some embodiments may also advantageouslyfurther include least one heat exchanger on the syringe, notably to cool the cement contained inside the syringe before being injected in the vertebra.

Advantageously, the heat exchanger on the syringe may be a passive heat exchanger, in order to keep the physical properties of the cement as constant as possible through the injection when not circulating in the pipe. Possibly or pre.era .v passive heat located on the syringe may include a sheath surrounding the syringe that is possibly or preferably filled with a eutectic fluid such as a gel.

Advantageously, the active and/or passive heat exchangers may be removable.

Advantageously, the injection device may also include a pressure sensor presenting a sensing area, the pressure sensor being located on a defined point of the cement pipe. Possibly or preferably, the sensing area of the pressure sensor may not be in direct contact with the cement.

Advantageously, the pressure of the cement in the pipe may be transmitted to the sensing area of the pressure sensor by an intermediary incompressible material, possibly or preferably water or an elastomeric material. Possibly or preferably, the cement pipe further may further include a sterile elastomeric membrane, able to transmit the cement pressure to the pressure sensor.

The injection device of some embodiments presents the major advantages:
to give the physician an enhanced control over the injection procedure,
to give the physician a pretty complete information concerning the cement and the in state, that allows the lection of the cement at a high viscosity in order to reduce the leakage risk, and
to optimize the cement working period.
to remotely monitor intra vertebral pressure.

### BRIEF DESCRIPTION OF THE DRAWINGS

Other features and advantages of some embodiments will become more clearly apparent on reading the following description, given with reference to the appended figures, which illustrate non-limiting examples of possible or preferable realizations (FIGS. 1 and 2) and also non limiting examples of different realizations of an injection device that may be used in the method of some embodiments:

FIG. 1 represents schematically the viscosity regulation scheme composed of three nested closed-loops for realizing the controlling e8) of the active heat exchanger of an injection device, according to a possible embodiment of the method,

FIG. 2 represents schematically the thermal loop that is nested in the viscosity loop illustrated on FIG. 1,

FIG. 3 represents a tridimensional CAD general view of an injection device that is used in the method of some embodiments as a whole,

FIG. 4 represents a schematic diagram of the injection device illustrated on FIG. 3,

FIG. 5 represents a CAD view of a master device that remotely controls the injection,

FIG. 6A represents a tridimensional CAD view of a heat exchanger with a thermal block,

FIG. 6B represents its corresponding cross-sectional schematic view,

FIG. 7 represents a principle diagram of a deported active heat exchanger put on a closed fluid circuit with a fluid-to-cement heat exchanger, according to an embodiment of some embodiments,

FIG. 8 represents a detailed view of the deported active heat exchanger illustrated on FIG. 7,

FIG. 9 represents a detailed view of the water-to-cement heat exchanger illustrated on FIG. 7,

FIG. 10 represents an exploded view of a sheath surrounding the syringe, according to an realization of some embodiments,

FIG. 11 represents a detailed view of the syringe with sheath illustrated on FIG. 10,

FIG. 12 represents a cross-sectional schematic view of an embodiment of a pressure sensor (located on the cement pipe) with a water-filled channel,

FIG. 13 represents a cross-sectional schematic view of another embodiment of a pressure sensor with an elastomer-filled channel,

FIG. 14 represents a cross-sectional schematic view of another embodiment of a pressure sensor with both an elastomer-filled channel and an elastomeric membrane.

For the sake of clarity, identical or similar elements have been referenced with identical reference symbols in all or most of the figures.

### DETAILED DESCRIPTION OF THE EXEMPLARY EMBODIMENTS

For purposes of understanding the principles of some embodiments, reference will now be made to the realizations illustrated in the drawings and the accompanying text.

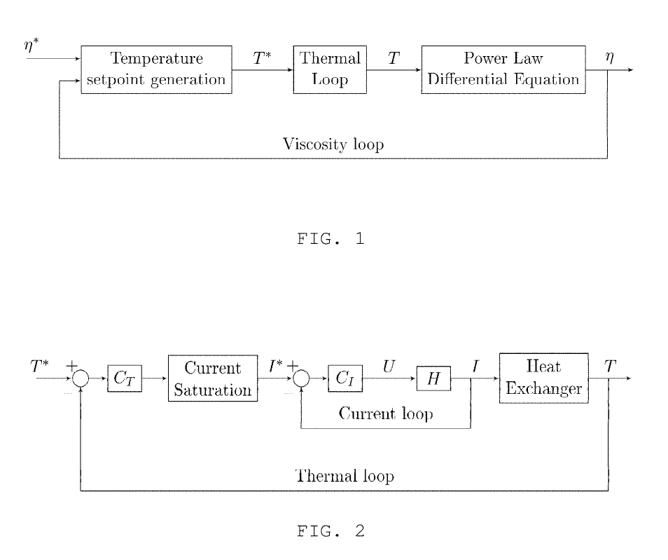

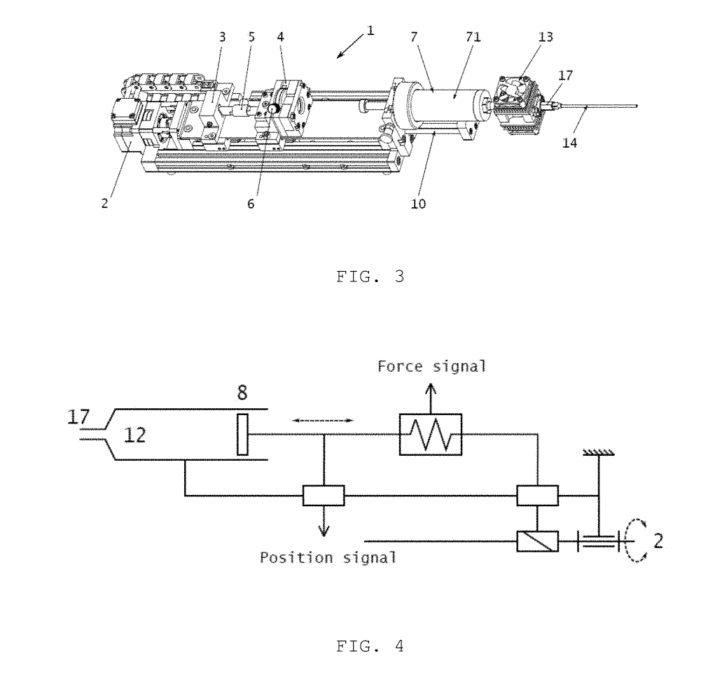

An advantage of the method of some embodiments is to control the cement viscosity. A regulation scheme composed of three nested closed-loops may be used for realizing the controlling e8) of the active heat exchanger of an injection device, as shown by FIGS. 1 and 2:

A. after defining for the bone cement to be injected the target viscosity η* to be reached or maintained, the target viscosity η* being included in the range [ηₘᵢₙ−ηₘₐₓ], ηₘᵢₙ being the minimal threshold viscosity of the cement which has to be reached for beginning the injection and ηₘₐₓ being the maximum threshold viscosity of the cement above which the injection is not possible anymore;

B. the set point temperature T*(t) associated to the target viscosity η* is computed according to the method of some embodiments (step e6) in the “temperature set point generation block” in the viscosity loop of FIG. 1,

C. the value of the set point temperature T*(t) is injected in the temperature regulation loop that is illustrated on FIG. 2, in which a temperature controller C_{T} uses the difference ε_{T} between the previously determined set point temperature T*(t) and the effective measured temperature T to compute the current reference I* of the active heat exchanger (usually or always using electrical energy as input),

D. the current reference I* is limited by a current saturation block,

E. the current is also controlled in a closed loop control (current loop), in which a current controller C_{T} uses the difference ε_{I} between the current reference I* and the effective input current I to compute the input voltage U of a power supply H driving the active heat exchanger (current loop).

Now concerning the injection device 1 that may be used in the method of some embodiments, FIG. 3 represents a tridimensional (3D) CAD general view of the entire injection device 1 of some embodiments as a whole. It is based on a quite straightforward design, using a ball screw linear axis to transform the rotation of the rotor of a high torque servo-motor 2 into a linear translation, in order to push on a high-pressure syringe 7. Two separated mobile carts are placed on the axis, with the first one (3) being motorized by the screw while the second one (4) is capable of moving along the axis freely. The two carts 3 and 4 are linked together through a force sensor 5 in order to measure the linear force running through the assembly 1. A hand maneuvered clamping device 6 has been placed on the free cart 4. It is used to grip a specific syringe piston rod 8 and has been equipped with a low force limit switch to provide an automatic approach during the setup phase. An incremental encoder has also been added between the free cart 4 and the base in order to provide a direct reading of the cart position without being affected by the potential backlash and the flexibility of the kinematic chain.

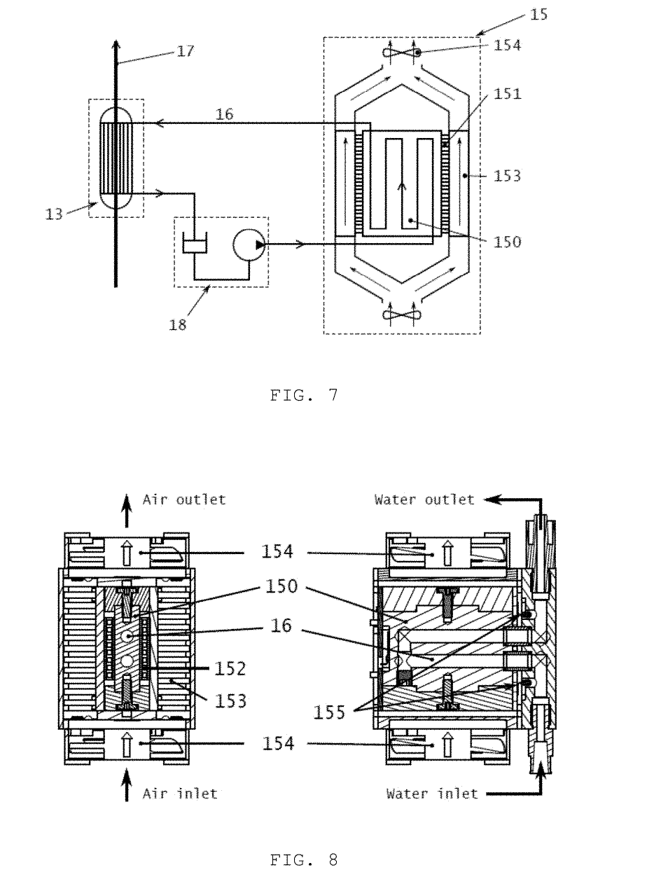

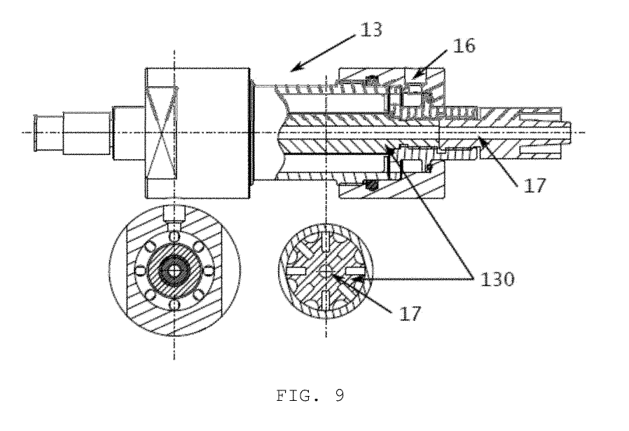

On the fixed part of the device, a mounting base has also been designed to support the syringe. The syringe itself is fixed to the mounting base by using a specific sheath that will be more precisely illustrated and detailed below (see FIGS. 6 and 7 and the corresponding accompanying paragraphs of the text). The syringe 7 is disposable because of the medical nature of its use.

FIG. 3 also shows a percutaneous needle 14 connected to the syringe 7 via a pipe 17. An active heat exchanger 13 such as a thermal block (see also FIGS. 4A and 4B) is located on the pipe 17) (surrounding the pipe 17) for the dynamic controlled heating and/or cooling of the cement 12 during the injection.

FIG. 4 represents a schematic diagram of the injection device shown on FIG. 3. The motor input 2 provide the displacement of the piston 8 that pushes the cement 12 in the cement channel 17. As shown of the diagram, both the position and force signal are provided by the system.

The overall dimensions of the injection device 1 as a whole are approximately 500×100×100 mm for a mass of approximately 5.5 kg. It can provide a service load of 2 kN. This can generate a pressure of about 100 bar on the cement 12 in the syringe 7, and will allow to inject a fluid with a viscosity up to 2000 Pa·s at a flow rate of 33 mm³/s considering the pressure drop of a 150×0 2.5 mm cylinder (equivalent to a typical cement near the end of its solidification injected in a typical large section injection needle plugged into a short channel).

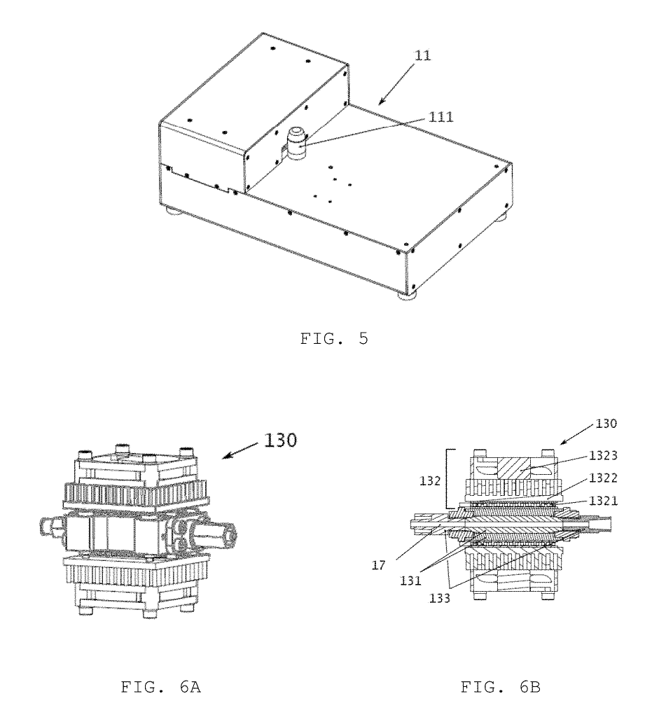

A master device 11, illustrated on FIG. 5, controls the injection remotely. In order to give to the physician the same feedback as he would have in a manual injection, the master device 11 can provide the force feedback of the pressure applied to the cement 12. Concerning the control of the injection itself, it is a flow rate control, which is more precise and practical than a volumetric control, generally provided by the current known manual systems. It then adds a design constraint to the master device 11 that should return to neutral position when the physician releases the interface because of safety issues. Besides that, the remote control also allows some flexibility as it may have some scaling or non-linear fitting both on the force-feedback and on the control signals, giving the possibility to customize easily both features with the experienced feedback of the practitioner.

The control of the injection is done via a rotating knob 111 located on the master device that returns the pressure information in the form of a force feedback. Should the physician release the knob during the injection, an integrated spring returns the interface in a neutral position.

As regards the active thermal regulation, which is realized in the method some embodiments by an active heat exchanger 13, FIGS. 6A and 6B show a first embodiment of an active heat exchanger 13 including or consisting of a unique thermal block 130. A thermal insulation (not shown on these figures) wraps the central part of the block 130. The thermal block is composed of
a central regulated block 131 that is crossed by the cement pipe 17.
two stacks 132, disposed symmetrically to the regulated block which include:
   two Peltier modules 1321,
   two heat sinks 1322,
   two fans 1323,
a thermal insulation (not shown on FIGS. 4A and 4B) wrapping the central block 131, which reduces the thermal exchanges between the regulated volume and the ambient air,
three temperature sensors (not shown on FIGS. 6A and 6B) placed respectively on the central block 131 and on both heat sinks 1322 to measure the temperature of the regulated part and to control both Peltier modules, and
two Luer-lock connections at the extremity of the channel 17 to plug the thermal block 130 to the syringe 7 on one side and to the needle 14 on the other side

The active thermal regulation may also be alternatively realized by a deported active heating/cooling heat exchanger 15 put on a closed water circuit 16 with a water-to-cement heat exchanger 13 located on the pipe 17 of the injection device 1, according to a second realization of some embodiments. Such a deported device allows a remote control of the viscosity of the cement during the intervention, thus protecting the radiologist during the cement injection phase by keeping her/him outside the radiation area.

FIG. 7 represents a schematic diagram of this closed water circuit 16 including the deported heat exchanger 15 and the water-to-cement heat exchanger 13 of the injection device 1. The water circulation is powered by a tanking/pumping device 18 placed on the closed loop 16. The advantage of this thermal regulation, in comparison with the thermal block of the first realization, is the possibility of doing the same regulation at a remote location, as the thermal block 130 of the first embodiment is both fragile, heavy and space consuming in a critical place such as the surgical area.

The deported heat exchanger 15 of FIGS. 7 and 8 (detailed section of FIG. 7) includes:
a heat transfer block 150 being crossed by a water circuit 16 connected to the water-to-cement heat exchanger 13,
   Peltier cells 152
   heat sinks 153, and
   fans 154.
   temperature sensors 155 placed on the water circuit 16.

As the orientation of the fan/sink couple has an impact on the performance on the heat sinks dissipation in free airflow, the fans 154 have been placed in a geometry designed to provide a more efficient forced airflow.

The water circuit 16 shown on FIG. 8 also includes a pumping system 18 that is able to work in reverse direction in order to purge the circuit 16 and thus, to avoid water leakage when the physician unplugs the exchanger 13 from the circuit 16.

The water-to-cement heat exchanger 13 shown on FIG. 9 is built around a finned block 130 whose task is to ensure a proper heat transfer between the cement pipe 17 and the water circuit 16. In this realization, it replaces the thermal block 13 presented in FIG. 1 on the cement pipe 17.

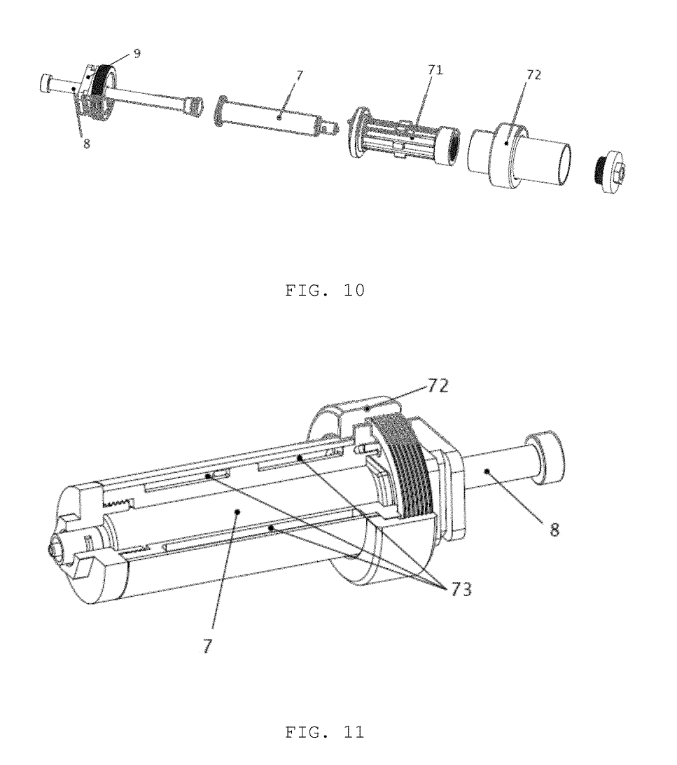

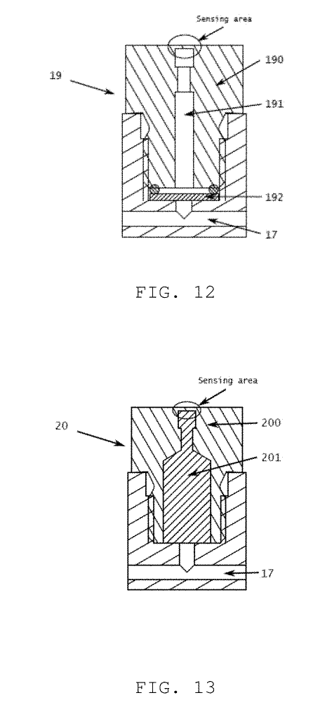

Now concerning the passive thermal exchange, FIGS. 10 and 11 represent a sheath 71 surrounding the syringe 7, according to a realization of some embodiments. In order to design this sheath, several constraints were taken in account:
it has to resist the service load that may rise up to 2 kN,
it should be able to passively exchange thermal energy as to maintain the cement stored within the syringe 7 with the lowest viscosity possible throughout the injection,
it has to be easily interfaced to the injection device 1 by having a dedicated interface 9 and by allowing a fast and easy locking of the syringe piston 8 to the free cart 4.

As such, the sheath 71 has been machined out of a 316L stainless steel in order to provide a high mechanical resistance and to resist to various chemical products, including biologic fluids and asepsis solutions. The sheath 71 provides some space around the syringe that may filled with an eutectic mixture known for its ability to exchange heat at constant temperatures, thus ensuring that the syringe 71 is kept cool during most of the injection.

The assembly (sheath) is also equipped with a fixation 9 at the back that interfaces with the mounting base 10 on the injector. A nut 72 and screw system is used to put in and extract the disposable syringe that contains the cement.

FIG. 10 shows the syringe 71, the syringe sheath 71 (partly disassembled) and the high-pressure piston 8, while

FIG. 11 shows a more detailed cutout of the sheath with the syringe 7 in it, where the room 73 for the eutectic gel is visible.

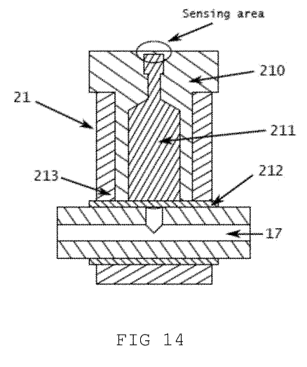

Now concerning pressure measurements of the cement along the cement pipe 17, FIGS. 12 to 14 are cross-sectional schematic views of different embodiments of pressure sensors 19, 20, 21 located on the pipe 17. In order to have a better control on and understanding of the injection procedure, it may be helpful to estimate the intravertebral pressure. This pressure may be used intraoperatively to detect failure during the procedure, such as pressure spikes or drops that may be symptomatic of clogging or leakage.

The best or better way to measure the intravertebral pressure would be to integrate a pressure sensor at the tip of the needle, but it would be very constraining in terms of design. Thus, in the frame of some embodiments, the value of this pressure is obtained indirectly, using a pressure measurement in the cement channel.

Considering the flow of a cement with varying rheological parameters K and n in a cylindrical pipe of length L_{vertebra} and of radius r, and given the known sensor position distant from the pipe inlet by a distance Lₛₑₙₛₒᵣ, the Poiseuille flow leads to equation (6):

$$Q = {( \frac{Δ P}{{L}_{sensor}} )}^{1 / n ( t )} {( \frac{r}{2 K ( t )} )}^{1 / n ( t )} ( \frac{π n ( t ) {r}^{3}}{3 n ( t ) + 1} ) \qquad ( 6 )$$

Assuming that the flow rate is high enough, the cement viscosity at the sensor is substantially equal to the cement at the outlet of the pipe. This provide then equation (7):

$${P}_{vertebra} = {P}_{o} ( 1 - \frac{{L}_{vertebra}}{{L}_{sensor}} ) + \frac{{L}_{vertebra}}{{L}_{sensor}} {P}_{i} \qquad ( 7 )$$

As shown by the equations, the knowledge of at least one pressure outside the injection pressure is mandatory. However, as pressure sensors are too expensive to be integrated as a disposable component, there is a need for a reusable pressure sensor that could be carried over several interventions. Also because of sterility issues, the sensor should not have any internal interface with the cement in order to be cleanable.

For this purpose, reusable pressure sensors have been developed in the frame of some embodiments, in which the sensing area of a standard pressure sensor is immersed in an incompressible fluid that would transfer the pressure.

According to a first advantageous embodiment of such a pressure sensor 19 (as shown on FIG. 10), it is based on a standard pressure sensor 190 whose channel 191 is filled with water. A flexible interface 192 separates the cement channel 17 and the sensor channel 191. The interface 192 can include or can consist of a cap made out of a flexible silicon compound, such a polydimethylsiloxane (PDMS).

According to a second advantageous embodiment of such a pressure sensor 20 (as shown on FIG. 11), it is based on a standard pressure sensor 200 whose channel is filled with an elastomeric compound 201 such as PDMS.

According to a third advantageous embodiment of such a pressure sensor 21 (as shown on FIG. 12), it is based on a standard pressure sensor 210 whose channel is filled with an elastomeric compound 211 such as PDMS. The cement pipe 17 is equipped with an elastomeric membrane 212, forming a measure point. The pressure sensor is mounted on a removable bracket 213 that may be plugged on the cement pipe 17, and then removed when the pipe 17 is disposed at the end of the intervention.

### LIST OF THE CITED REFERENCES

[1] A. Gangi, S. Guth, J. Imbert, H. Marin, and J.-L. Dietemann, “Percutaneous vertebroplasty: indications, technique, and results.” Radiographics, vol. 23, March 2003.
[2] US 2013/0190680 of Baroud: US patent application filed on Mar. 8, 2013 by the SOCPRA S.E.C and published on Jul. 25, 2013.
[3] US 2009/0062808 of Wolf: US patent application filed on March 2008 by Wolf (as inventor and applicant) and claiming the priority of a provisional application dated Sep. 5, 2007, and published on Mar. 5, 2009.
[4] U.S. Pat. No. 8,523,871 of Truckai et al.: US granted patent filed on Apr. 3, 2008 by Truckai et al. (as inventors and applicants) and claiming the priority of four provisional applications dated Apr. 3, 2007, and granted on Oct. 9, 2008.
[5] N. Lepoutre, G. Bara, L. Meylheuc, et B. Bayle, “Phase Space Identification Method for Modeling the Viscosity of Bone Cement”, in Control Conference (ECC), 2016 European, Juin-Juillet 2016.

1. A method for a dynamic control of the viscosity of an orthopedic bone cement during curing by acting on a bone cement temperature in percutaneous vertebroplasty, within an injection device that includes a syringe, a percutaneous needle connected to the syringe via a pipe, including an active heat exchanger, the method comprising:
A. defining the time tₒ, time at which the radiologist starts the mixing process of the bone cement;
B. filling the syringe with the prepared bone cement;
C. defining for the bone cement a target viscosity η* to be reached or maintained, the target viscosity η* being in the range [ηₘᵢₙ−ηₘₐₓ], ηₘᵢₙ being the minimal threshold viscosity of the cement which has to be reached for beginning the injection and ηₘₐₓ being the maximum threshold viscosity of the cement above which the injection is no longer possible;
D. beginning the injection of the bone cement into the vertebra;
E. at instant t during the injection:
e1) measuring an effective temperature T of the bone cement at an outlet of the active heat exchanger and measuring an effective temperature Tᵢ of the bone cement at an inlet of the active heat exchanger;
e2) computing the pressure drop ΔP=Pₒ−Pᵢ along the pipe between the outlet of the syringe and a given intermediate point, Pₒ being the pressure measured at the outlet of the syringe and Pᵢ being the pressure measured at the given intermediate point on the pipe, the length between those two points being denoted as Lₛₑₙₛₒᵣ;
e3) computing a flow rate Q of the bone cement in the pipe;
e4) computing a shear rate \dot{γ}ₚ at the wall of the pipe as a function of the flow rate Q, the cross-section dimensions of the pipe and the intrinsic physical parameters of the cement;
e5) calculating the instant viscosity
η(t,T,\dot{γ}_{P}) if Q is nonzero, as a function of time t, temperature T, pressure drop ΔP and shear rate \dot{γ}ₚ, itself function of the flow rate Q;
η₀(t,T) if Q has a zero value, as a function of time t and temperature T.
e6) computing a set point temperature T(*)t associated to the target viscosity η* and the instant viscosity η, η* being function of the flow rate Q and the time t;
e7) calculating the difference ε_{T} between the previously determined set point temperature T(*)t and the effective temperature at the outlet of the heat exchanger T;
e8) controlling the cooling or the heating of the bone cement throughout the control of the active heat exchanger as a function of ε_{T};
F. at instant t+Δt, repeating step E until the end of the injection, unless the instant viscosity η(t,T,\dot{γ}_{P}) and/or η₀(t,T) has reached the maximum threshold viscosity ηₘₐₓ.

2. The method according to claim 1, wherein step F further comprises the redefinition of the target viscosity η* before repeating step E until the end of the injection, unless the instant viscosity η(t,T,\dot{γ}_{P}) and/or η₀(t,T)has reached the maximum threshold viscosity ηₘₐₓ.

3. The method according to claim 1, wherein the step e2) of computing the pressure drop ΔP is realized between the outlet of the syringe and the outlet of the needle.

4. The method according to claim 1, wherein the step e2) of computing the pressure drop ΔP is realized between the outlet of the syringe and the outlet of the active heat exchanger.

5. The method according to claim 1, wherein the instant viscosity η(t,T,\dot{γ}ₚ), if the flow rate is nonzero, is calculated according to modified Power Law as defined by formula (2) in the case of a pipe having a cylindrical geometry of radius r:
$$η ( t , T , {\dot{γ}}_{p} ) = {a}_{{T}_{0}} ( T ) K ( t ) {( {a}_{{T}_{0}} ( T ) {\dot{γ}}_{p} )}^{n ( t ) - 1} \\ with \\ {a}_{{T}_{0}} ( T ) = exp ( \frac{- {E}_{a}}{R} ( \frac{1}{T} - \frac{1}{{T}_{0}} ) ) \qquad ( 2 )$$
with:
Eₐ being the activation energy in J.mol⁻¹,
T being the effective temperature of the bone cement at the outlet of the active heat exchanger,
T₀ being a reference temperature at which the viscosity η_{□}□ is known,
R being the gas constant,
n(t) being the flow index of the bone cement at the current time t, n is either a known constant or defined as a function of t₀ and t.
being the shear rate at the wall of the pipe being given by formula (3):
$${\dot{γ}}_{p} = \frac{Q}{π {r}^{3}} \frac{3 n ( t ) + 1}{n ( t )} \qquad ( 3 )$$
with r being the radius of the pipe.
K(t) being given by formula (4):
$$Q = {( \frac{Δ P}{{L}_{sensor}} )}^{1 / n ( r )} {( \frac{r}{2 K ( t )} )}^{1 / n ( t )} ( \frac{π n ( t ) {r}^{3}}{3 n ( t ) + 1} ) \qquad ( 4 )$$

6. The method according to claim 1, wherein the instant viscosity η(t) is calculated according to the differential equation (5):
\dot{η}(t,T,\dot{γ}ₚ)=f(η(t,T,\dot{γ}ₚ)) (5)
wherein the time derivative fi of the viscosity is defined as a function the instant viscosity η.

7. The method according to claim 1, wherein the set point temperature T(*)t is calculated according to a chosen control strategy either via
$${argmin }_{T} \dot{η}$$
or using the inverse solution of equation (5).

8. The method according to claim 1, wherein the step of measuring the flow rate Q of the bone cement in the pipe comprises a step of measuring a moving speed Vₚᵢₛₜ of the piston of the syringe, the piston being driven to vary the volume of the cement in the syringe, the volumetric flow Q being then given by Q=Vₚᵢₛₜ.π.r².

9. The method according to claim 1, wherein the controlling e8) of the active heat exchanger realizes the cooling or heating of the bone cement as a function of ε_{T} throughout a temperature regulation scheme composed of two nested closed loops, where:
a temperature controller C_{T} uses the difference ε_{T} between the previously determined set point temperature T(*)t and the effective temperature T to compute the current reference I* of the active heat exchanger, the current reference I* being limited by a current saturation block,
a current controller C_{I} uses the difference ε_{I} between the current reference I* and the effective input current I to compute the input voltage U of a power supply H driving the active heat exchanger.

10. The method according to claim 4, wherein the intravertebral pressure P_{vertebra} is computed according to formula (1):
$${P}_{vertebra} = {P}_{o} ( 1 - \frac{{L}_{vertebra}}{{L}_{sensor}} ) + \frac{{L}_{vertebra}}{{L}_{sensor}} {P}_{i} \qquad ( 1 )$$
with:
L_{vertebra} being the length comprised between the outlet of the syringe and the outlet of the needle.

11. The method according to claim 2, wherein the step e2) of computing the pressure drop ΔP is realized between the outlet of the syringe and the outlet of the needle.

12. The method according to claim 2, wherein the step e2) of computing the pressure drop ΔP is realized between the outlet of the syringe and the outlet of the active heat exchanger.

13. The method according to claim 2, wherein the instant viscosity η(t,T,\dot{γ}ₚ), if the flow rate is nonzero, is calculated according to modified Power Law as defined by formula (2) in the case of a pipe having a cylindrical geometry of radius r:
$$η ( t , T , {\dot{γ}}_{p} ) = {a}_{{T}_{0}} ( T ) K ( t ) {( {a}_{{T}_{0}} ( T ) {\dot{γ}}_{p} )}^{n ( t ) - 1} \\ with \\ {a}_{{T}_{0}} ( T ) = exp ( \frac{- {E}_{a}}{R} ( \frac{1}{T} - \frac{1}{{T}_{0}} ) ) \qquad ( 2 )$$
with:
Eₐ being the activation energy in J.mol⁻¹,
T being the effective temperature of the bone cement at the outlet of the active heat exchanger,
T₀ being a reference temperature at which the viscosity η_{□}□ is known,
R being the gas constant,
n(t) being the flow index of the bone cement at the current time t, n is either a known constant or defined as a function of t₀ and t.
\dot{γ}ₚ being the shear rate at the wall of the pipe being given by formula (3):
$${\dot{γ}}_{p} = \frac{Q}{π {r}^{3}} \frac{3 n ( t ) + 1}{n ( t )} \qquad ( 3 )$$
with r being the radius of the pipe.
K(t) being given by formula (4):
$$Q = {( \frac{Δ P}{{L}_{sensor}} )}^{1 / n ( r )} {( \frac{r}{2 K ( t )} )}^{1 / n ( t )} ( \frac{π n ( t ) {r}^{3}}{3 n ( t ) + 1} ) \qquad ( 4 )$$

14. The method according to claim 2, wherein the instant viscosity η(t) is calculated according to the differential equation (5):
\dot{η}(t,T,\dot{γ}ₚ)=f(η(t,T,\dot{γ}ₚ)) (5)
wherein the time derivative of the viscosity is defined as a function the instant viscosity η.

15. The method according to claim 3, wherein the instant viscosity η(t) is calculated according to the differential equation (5):
\dot{η}(t,T,\dot{γ}ₚ)=f(η(t,T,\dot{γ}ₚ))
wherein the time derivative 1) of the viscosity is defined as a function the instant viscosity η.

16. The method according to claim 4, wherein the instant viscosity η(t) is calculated according to the differential equation (5):
\dot{η}(t,T,\dot{γ}ₚ)=f(η(t,T,\dot{γ}ₚ))
wherein the time derivative 3 of the viscosity is defined as a function the instant viscosity η.

17. The method according to claim 2, wherein the set point temperature T(*)t is calculated according to a chosen control strategy either via
$${argmin }_{T} \dot{η}$$
or using the inverse solution of equation (5).

18. The method according to claim 3, wherein the set point temperature T(*)t is calculated according to a chosen control strategy either via
$${argmin }_{T} \dot{η}$$
or using the inverse solution of equation (5).

19. The method according to claim 4, wherein the set point temperature T(*)t is calculated according to a chosen control strategy either via
$${argmin }_{T} \dot{η}$$
or using the inverse solution of equation (5).

20. The method according to claim 5, wherein the set point temperature T(*)t is calculated according to a chosen control strategy either via
$${argmin }_{T} \dot{η}$$
or using the inverse solution of equation (5).
